(19) United States
(12) Plant Patent Application Publication
(10) Pub. No.: US 2013/0139286 P1
(43) Pub. Date: May 30, 2013

(54) Strawberry plant named 'Cupcake'

(50) ;

(71) Applicant: John Larse, Aptos, CA (US)

(72) Inventor: John Larse, Aptos, CA (US)

(21) Appl. No.: 13/373,705

(22) Filed: Nov. 28, 2011

(51) Int. Cl. A01H 5/00

(52) U.S. Cl. PLT/208

(57) ABSTRACT

The present invention provides a new and distinct strawberry variety designated as ‘Cupcake’ (a.k.a., ‘107615’).

### LATIN NAME OF GENUS AND SPECIES

Fragaria×ananassa

### VARIETAL DENOMINATION

‘Cupcake’ (a.k.a. ‘107615’)

### BACKGROUND OF THE INVENTION

The present invention relates to a new and distinct early day-neutral strawberry variety designated as ‘Cupcake’ (a.k.a. ‘107615’). This new variety is the result of a controlled-cross between a female parent cultivar designated ‘102213’ and a male parent cultivar designated ‘1581’ (both unpatented, proprietary cultivars) made by the inventor and first fruited in Watsonville, Calif. growing fields.

Following selection and during testing, the plant was originally designated ‘107615’ and subsequently named ‘Cupcake’. The new variety of ‘Cupcake’ was asexually reproduced via runners (stolons) by the inventor at Watsonville, Calif. Asexual propagules from the original source have been tested in Watsonville growing fields and to a limited extent, grower fields in low and high elevation. The properties of this variety were found to be transmissible by such asexual reproduction. The cultivar is stable and reproduces true to type in successive generations of asexual reproduction.

### BRIEF SUMMARY OF THE INVENTION

This invention relates to a new and distinctive early producing day-neutral type cultivar designated as ‘Cupcake’. It is primarily adapted to the climate and growing conditions of the central coast of California. This region provides the necessary temperatures required for it to produce a strong vigorous plant and to remain in fruit production from April through October. The nearby Pacific Ocean provides the needed humidity and moderate day temperatures and evening chilling to maintain fruit quality for the production months.

The following traits in combination distinguish strawberry variety ‘Cupcake’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The produced fruit is attractive and of excellent quality. Produced fruit is pink red to light-red at maturity, medium to large in size, firm to very firm, conical to cylindrical in shape with a smooth surface lacking creases. Seeds are slightly sunken below the surface.

‘Cupcake’ is primarily adapted to the growing conditions of the central coast of California, characterized by foliage small to medium in size and light to medium green in color. Production generally begins in April and produces continually into October with 40% to 50% of its production in the months of August through September.

### DESCRIPTION OF THE DRAWINGS

The accompanying color photographs depict various characteristics of the ‘Cupcake’ cultivar at various stages of development as nearly true as possible to make color reproductions.

FIG. 1 shows the plant in its production stage bearing fruit.

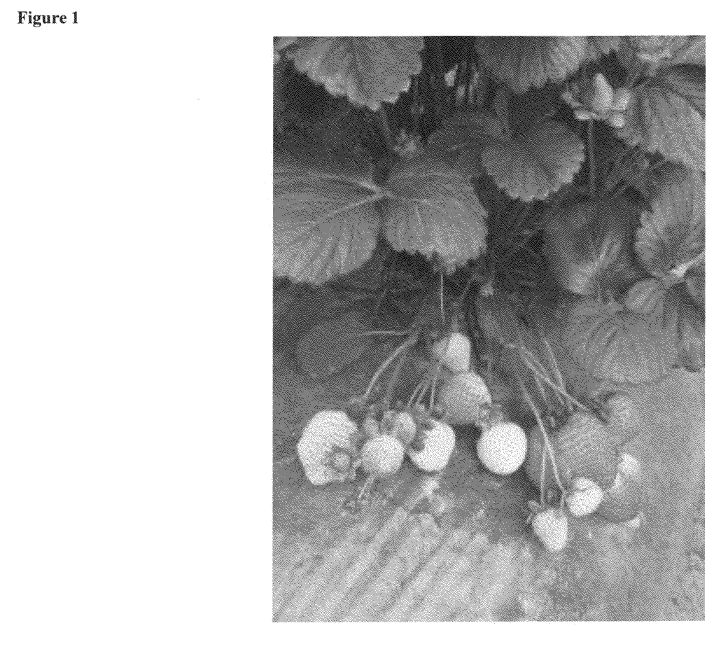

FIG. 2 shows the plant with flower and runners.

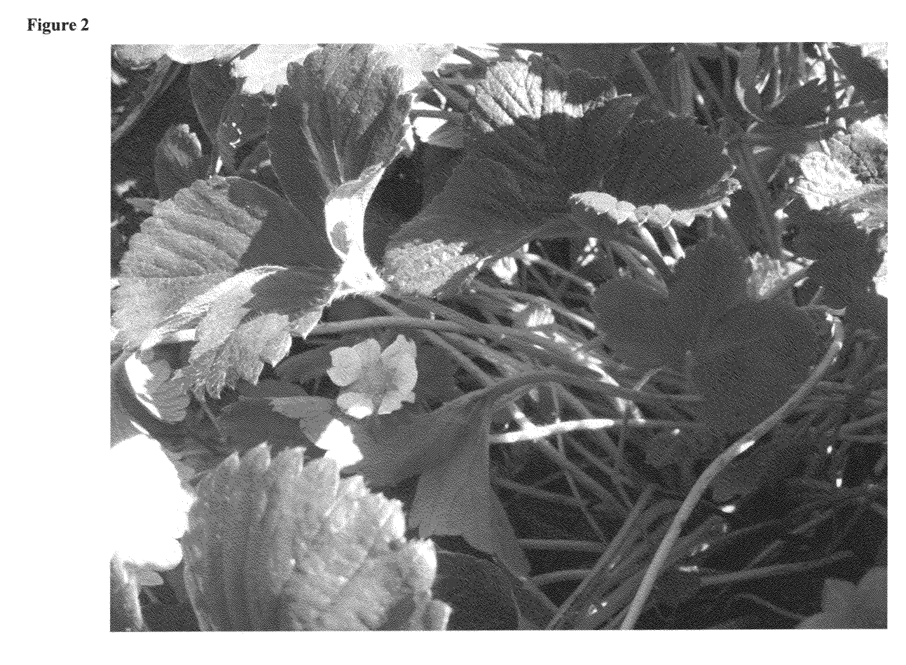

FIG. 3 shows typical leaves in the production cycle.

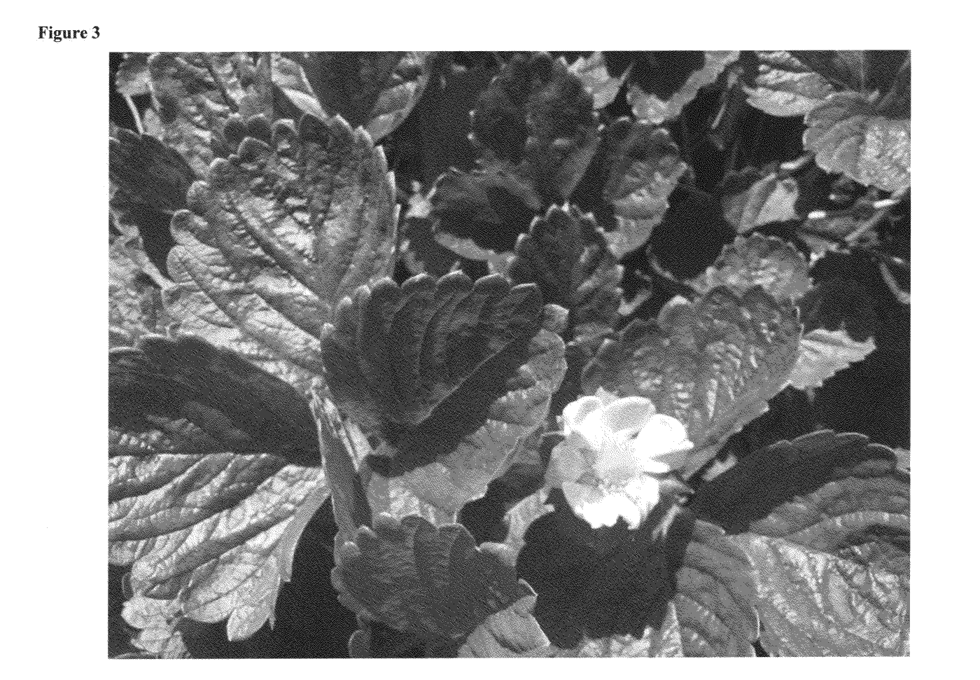

FIG. 4 shows ‘Cupcake’ plants in the field.

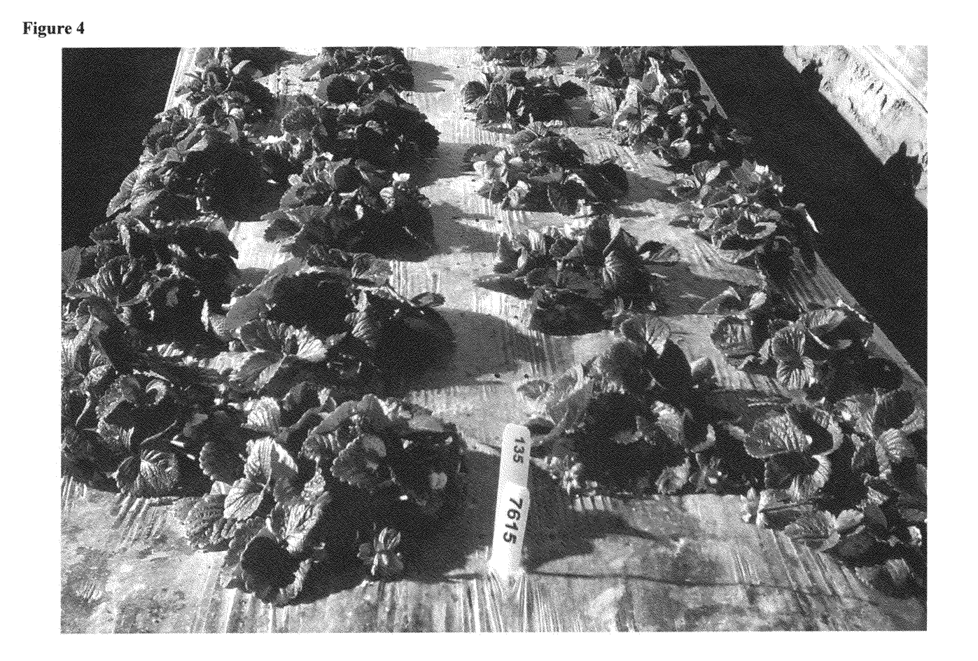

FIG. 5 shows the plant with unripe fruit and runners.

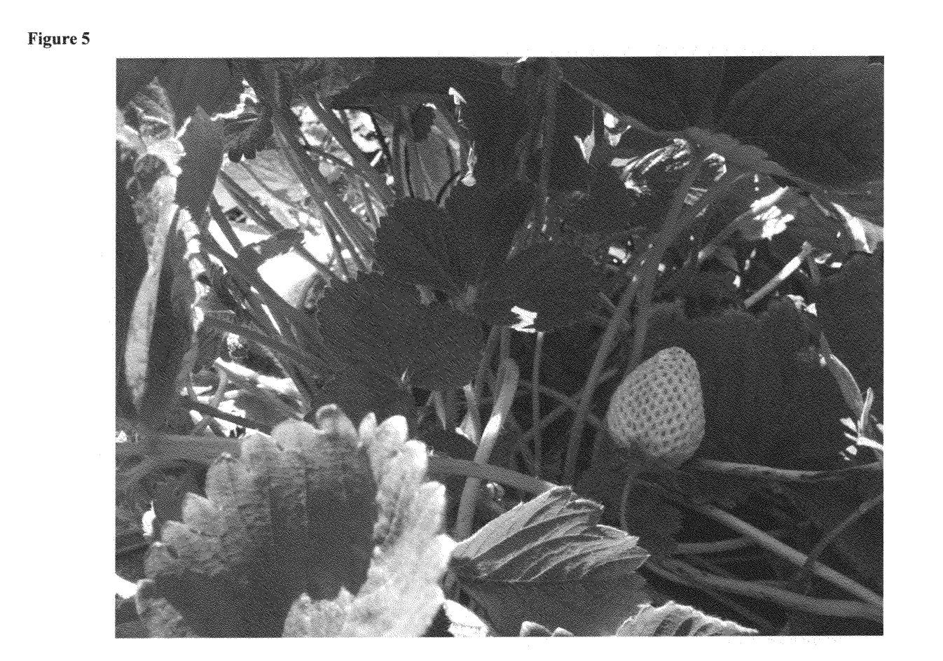

FIG. 6 shows sliced ‘Cupcake’ fruit.

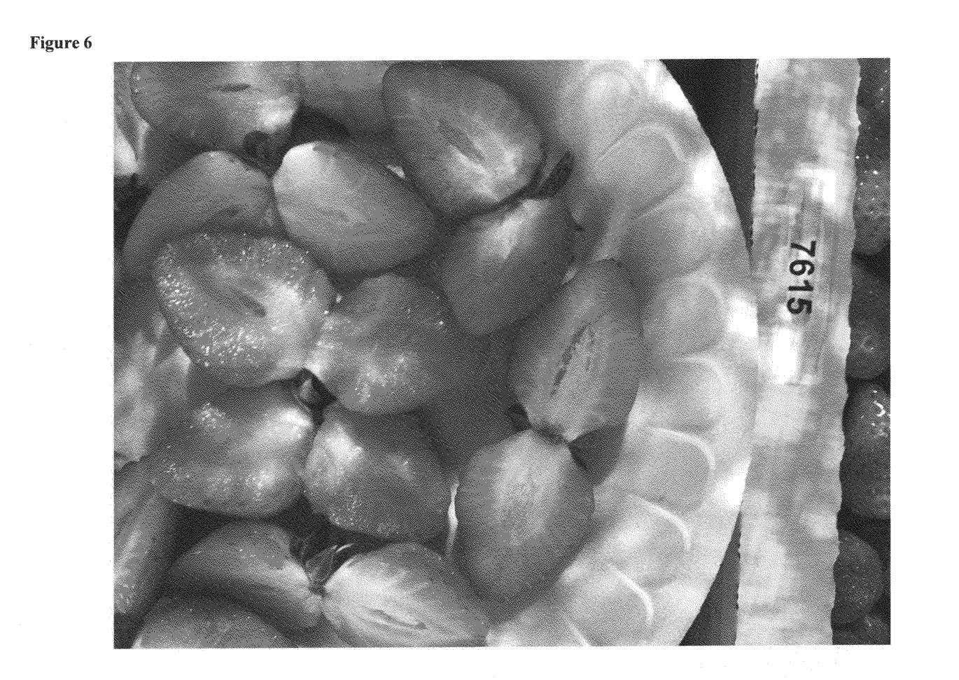

FIG. 7 shows ripe and near-ripe ‘Cupcake’ fruits.

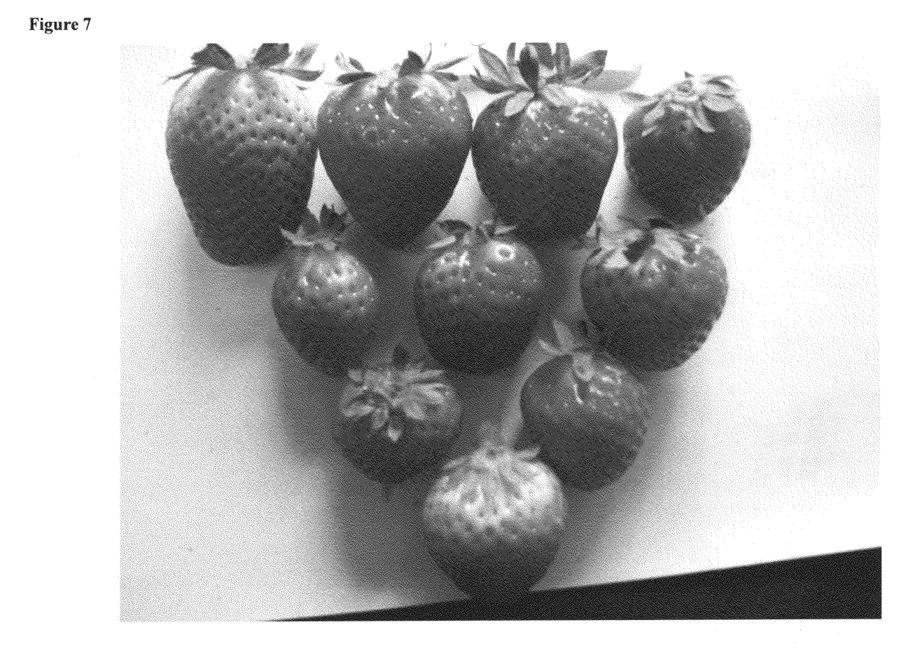

### DETAILED BOTANICAL DESCRIPTION

This invention relates to a new and distinctive day-neutral (ever-bearing) type cultivar designated as ‘Cupcake’, but may be considered a weak day-neutral in that it is somewhat similar to a short day. When grown in moderate, coastal climates “Cupcake’ loads up with larger fruit in the year before the summer solstice, but with an extended harvest period of fairly heavy volume into autumn season. ‘Cupcake's fruit is medium to large in size, that is relatively firm to very firm, mostly conical to cylindrical shape and some wedge shape with seeds slightly sunken below the surface.

Among the characteristics that distinguish the new variety from the other closely related varieties are the timing of fruiting, plant vigor, position of the inflorescences, leaf morphology, leaf size, leaf density, leaf color, and disease and pest resistance.

‘Cupcake’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.
Botanical classification: ‘Cupcake’ is a fertile hybrid derived from a cross.
Common name: Garden strawberry
General description:
   Plant habit.—moderate, coastal climates.
Flowering, fruiting and production characteristics: The flowers are perfect, petals are pure white and rounded and entire; and anthers and receptacle are bright yellow. The calyx position is generally centered with sepals that are light to medium green in color.
Foliar characteristics for ‘Cupcake’: Plant size is short to medium in stature. The shape of the base is obtuse and light to medium in density. Foliar color is medium to dark green on the adaxial surface of the leaf. The abaxial surface of the leaf is light silver-green to silver-green. No bract leaves were observed in samples. The shape of the leaf is slightly concave and flat Leaf length slightly exceeds the width. Each leaflet has three leaves. The leaves are moderately to highly glossy.
Flower and fruit characteristics for ‘Cupcake’: The average number of petals per flower is 6 with a range of 5-7 petals. The positioning of the inflorescence is even with or slightly below foliage. The flowers are mostly medium small to medium in size. The plant produces mostly conical fruit with some long cones as well as long wedges and the fruit color is medium opacity red and light gloss. Seed position on fruit is somewhat indented. The fruit's flavor is consistently good or better than good throughout the season.
Variety identification testing for ‘Cupcake’: No matches to existing cultivars were found using SSR marker testing in a commercial database search.

1. A new and distinct cultivar of strawberry plant named ‘Cupcake’ substantially as shown and described herein.
